(19) United States
(12) Patent Application Publication
(10) Pub. No.: US 2026/0102280 A1
(43) Pub. Date: Apr. 16, 2026

(54) THERAPEUTIC PACK CONFORMED FOR APPLICATION TO HUMAN AND ANIMAL BODY PARTS

(71) Applicant: PurHeart, LLC, San Francisco, CA (US)

(72) Inventor: Maria Sheila Garcia, San Francisco, CA (US)

(21) Appl. No.: 19/338,241

(22) Filed: Sep. 24, 2025

Related U.S. Application Data
(60) Provisional application No. 63/707,889, filed on Oct. 16, 2024.

(51) Int. Cl. A61F 7/03; A61F 7/02; A61F 13/00; A61F 13/02; A61K 31/7036; A61K 36/28; A61K 36/53; A61K 36/736; A61K 38/12

(52) U.S. Cl. CPC A61F 7/03; A61F 13/00063; A61F 13/023; A61K 31/7036; A61K 36/28; A61K 36/53; A61K 36/736; A61K 38/12; A61F 2007/022; A61F 2007/0226; A61F 2007/0244; A61F 2007/0261; A61F 2013/00187; A61F 2013/00285

(57) ABSTRACT

A very thin cold and/or heat therapy gel pack with a thickness of approximately 5 mm is designed to conform to the complex contours of a human or animal body through the strategic placement of seams (referred to herein as “voids”) that allow the gel packs to bend and conform in anatomically correct places to a particular body part. The gel packs may attach to the user via hook and loop closures, buckle closures, strap closures, and adhesive closures. For cold therapy, the gel packs may attach to the user via a pressure sensitive medical adhesive, such as medical tape. Where single-use gel packs are necessary for wound healing, a sterile dressing pad may be embedded in the gel packs. The voids as described herein may also be used on disposable therapy packs that do not contain a gel, such as instant cold therapy packs and instant dry heat packs.

### TECHNICAL FIELD

The present disclosure relates generally to therapeutic packs, and more specifically to very thin cold or heat therapeutic packs that are designed to conform to the complex contours of human and animal body parts.

### BACKGROUND OF THE DISCLOSURE

Therapeutic packs are used to provide hot and/or cold therapy to an area of a human or animal body that has been subject to injury, aches, and pain. When therapeutic packs are used as cold packs, the material of the conventional cold packs used in the art do not conform readily to the complex curves of a human and animal body. When therapeutic packs are used as heat packs, the material of the conventional heat packs can become slack and fail to hold their shape. In view of the foregoing, there remains a need in the art for a cold and/or heat therapeutic pack that is able to conform to the complex curves of human or animal bodies.

### SUMMARY OF THE DISCLOSURE

The present invention overcomes the need in the art by providing a very thin therapeutic pack with strategically placed voids that allow the pack to flex and bend in an anatomical fashion to the body part to which the gel pack is being applied.

In one embodiment, the present invention relates to a gel pack comprising: a gel material, and a shell material enclosing the gel material, wherein the gel pack is a cold or heat therapy pack that includes at least one void area free of the gel material, wherein the at least one void area provides the gel pack with flexibility that allows the gel pack to closely conform to an anatomical area or body part of a human or animal user.

In another embodiment, the at least one void area comprises linear voids.

In a further embodiment, the at least one void area comprises non-linear voids.

In another embodiment, the at least one void area comprises a combination of linear and non-linear voids.

In a further embodiment, the width of the at least one void area will range from 1-5 mm and the length of the at least one void area will range from 2-10 cm.

In another embodiment, the shell material has a thickness of 1-2 mm without the filling material.

In a further embodiment, the thickness of the cold or heat therapy pack when filled with the filling material is 5-10 mm.

In another embodiment, the filling material is a gel comprising a thickening agent, water, and an anti-freezing agent.

In a further embodiment, the thickening agent is selected from the group consisting of carboxymethyl cellulose, hydroxyethyl cellulose, sodium polyacrylate silica gel, and combinations thereof.

In another embodiment, the anti-freezing agent is selected from the group consisting of tri-methylene glycol, diethylene glycol, ethylene glycol, propylene glycol, butanediol, glycerol, and combinations thereof.

In a further embodiment, the thickening agent is carboxymethyl cellulose and the anti-freezing agent is tri-methylene glycol.

In another embodiment, the shell material is selected from the group consisting of polypropylene, polyvinyl chloride, polyethylene, polycarbonate, polytetrafluoroethylene, nylon, acrylonitrile butadiene styrene, and combinations thereof.

In a further embodiment, the shell material is polyvinyl chloride.

In another embodiment, the gel pack is secured to the anatomical area or body part of the human or animal user with at least one set of closures.

In a further embodiment, the at least one set of closures are selected from the group consisting of hook and loop closures, buckle closures, strap closures, adhesive closures, and combinations thereof.

In another embodiment, the shell material comprises a dressing pad on one side of the gel pack, wherein the side of the gel pack with the dressing pad is applied to a wound on the anatomical area or the body part of the human or animal user.

In a further embodiment, the gel pack is designed for application to an animal user, wherein the dressing pad is saturated with oregano oil and/or almond oil.

In another embodiment, the gel pack is designed for application to a human user, wherein the shell material comprises a medical grade silicone tape sheet on one side of the gel pack, wherein the side of the gel pack with the medical grade silicone sheet is applied to an area of a human user that requires scar maintenance.

In a further embodiment, the gel pack is designed to fit into a pocket of a compression garment, wherein the compression garment is designed to be worn around the anatomical area or the body part of the human or animal user.

In another embodiment, the shell material comprises a pressure sensitive medical adhesive on one side of the gel pack, wherein the gel pack adheres to the anatomical area or the body part of the human or animal user via the medical adhesive.

In a further embodiment, the pressure sensitive medical adhesive is medical tape.

In another embodiment, the pressure sensitive medical adhesive comprises a therapeutic agent selected from the group consisting of antibiotic agents, anti-inflammatory agents, pain relieving agents, anesthetics, and combinations thereof.

In a further embodiment, the therapeutic agent is the anti-inflammatory and pain-relieving agent known as Arnica montana.

In another embodiment, the antibiotic agent is selected from the group consisting of neomycin, bacitracin, polymyxin, and combinations thereof.

In a further embodiment of the present invention, the therapeutic pack is an instant cold therapy pack comprising a plastic pouch with a first compartment containing an endothermic salt and a second compartment containing water, wherein the first and second compartments are separated by a thin plastic barrier, wherein squeezing of the pouch ruptures the thin plastic barrier causing the water and endothermic salt to mix and form an endothermic reaction that lowers the temperature of the therapeutic pack, wherein the first and/or second compartments of the pouch comprise at least one void area free of the water and/or the endothermic salt, wherein the at least one void area provides the instant cold therapy pack with flexibility that allows the instant cold therapy pack to closely conform to an anatomical area or body part of a human or animal user.

In another embodiment of the present invention, the therapeutic pack is an instant dry heat therapy pack comprising an airtight and waterproof sealed outer pouch and an air-permeable inner pouch, wherein the air-permeable inner pouch comprises a filling that includes iron powder, wherein unsealing of the outer pouch exposes the iron powder to air, wherein reaction of the iron powder and oxygen from the air forms an oxidation reaction that raises the temperature of the therapeutic pack, wherein the air-permeable inner pouch comprises at least one void area free of the filling, wherein the at least one void area provides the instant dry heat therapy pack with flexibility that allows the instant dry heat therapy pack to closely conform to an anatomical area or body part of a human or animal user.

Additional embodiments and/or aspects of the disclosure will be provided, without limitation, in the detailed description of the disclosure that is set forth below.

### BRIEF DESCRIPTION OF THE DRAWINGS

FIGS. 1A and 1B are schematic diagrams of an exemplary cold and heat therapeutic half-leg gel pack for application to a human leg.

FIGS. 2A and 2B are photographs of the half-leg gel pack of FIGS. 1A and 1B.

FIGS. 3A and 3B are photographs of a gel pack for application to the upper leg of a user.

FIGS. 4A and 4B are photographs of one embodiment of a gel pack for application to the knee of a user.

FIGS. 5A and 5B are photographs of another embodiment of a gel pack for application to the knee of a user.

FIGS. 6A and 6B are photographs of a gel pack for application to the lower leg of a user.

FIGS. 7A and 7B are photographs of a gel pack for application to the ankle of a user.

FIGS. 8A and 8B are photographs of a gel pack for application to the whole foot of a user.

FIGS. 9A and 9B are photographs of a gel pack for application to half of the foot of a user.

FIGS. 10A and 10B are photographs of a gel pack for application

FIGS. 11A and 11B are photographs of a gel pack for application to the toes of the foot of a user.

FIGS. 12A and 12B are photographs of a gel pack for application to the hand of a user.

FIGS. 13A and 13B are photographs of a gel pack for application to the palm of the hand of a user.

FIGS. 14A and 14B are photographs of a gel pack for application to the hand and wrist of a user.

FIGS. 15A and 15B are photographs of a gel pack for application to the elbow of a user.

FIGS. 16A and 16B are photographs of a gel pack for application to the upper arm of a user.

FIGS. 17A and 17B are photographs of a gel pack for application to the entire arm of a user.

FIGS. 18A and 18B are photographs of a gel pack for application to the cheeks of a user.

FIGS. 19A and 19B are photographs of a gel pack for application to the lower face of a user.

FIGS. 20A and 20B are photographs of a gel pack for application to the back neck, shoulders, upper back, and upper chest area of a user.

FIGS. 21A-21D show an oval medical adhesive dressing pad gel pack for application to the nose of a user.

FIGS. 22A-22C show a medical adhesive dressing pad gel pack for application to the neck and throat of a user.

FIGS. 23A-23C show a medical adhesive dressing pad gel pack for application to the neck and ear of a user.

FIGS. 24A-24C show a medical adhesive dressing pad gel pack for application to the neck of a user.

FIGS. 25A-25C show a medical adhesive dressing pad gel pack for application to the spine and back of a user.

FIGS. 26A-26E show various medical adhesive gel packs that may be equipped with a dressing pad.

FIGS. 27A-27C show one embodiment of a medical adhesive gel pack for application to the head/scalp of a user.

FIGS. 28A-28C show another embodiment of a medical adhesive gel pack for application to the head/scalp of a user.

FIGS. 29A-29C show a medical adhesive gel pack for application to the breast and underarm of a user.

FIG. 30 shows a compression chest garment for application to a human user with pockets that contain one or more of the gel packs described herein.

FIGS. 31A and 31B show a cooling gel pack vest for application to the back of a feline or canine animal.

FIGS. 32A and 32B show a cooling gel pack suit for application around the trunk of a feline or canine animal.

FIGS. 33A and 33B show a cooling belly gel pack for application to the underside of a feline or canine animal that has undergone a spaying or neutering procedure.

FIGS. 34A and 34B show a collar gel pack for application to the base of the neck of a feline or canine animal.

FIGS. 35A and 35B show a muzzle gel pack for application to the mouth of a feline or canine animal to relieve dental or oral pain.

FIGS. 36A and 36B show an eye gel pack for application to a feline or canine animal that has undergone an eye procedure.

### DETAILED DESCRIPTION OF THE DISCLOSURE

Set forth below is a description of what are currently believed to be preferred embodiments and/or aspects of the disclosure. Any alternates or modifications in function, purpose, or structure are intended to be covered by the appended claims. As used in this specification and the appended claims, the singular forms “a,” “an,” and “the” include plural referents unless the context clearly dictates otherwise. The terms “comprise,” “comprised,” “comprises,” and/or “comprising,” as used in the specification and appended claims, specify the presence of the expressly recited components, elements, features, and/or steps, but do not preclude the presence or addition of one or more other components, elements, features, and/or steps.

Described herein are therapeutic packs for cold and/or heat therapy that are intended to be customized to fit a variety of human or animal body parts of different sizes and shapes. The therapeutic cold and/or heat packs are very thin and include voids, i.e., areas without the filling material, that allow the therapeutic packs to anatomically conform to various body parts.

As used herein, the term “therapeutic pack” refers to all packs described herein, including gel packs, instant cold packs, and instant dry heat packs. All therapeutic packs described herein are skin-safe, non-toxic, and resistant to leakage and material breakdown.

As used herein, the term “animal” refers to any warm-blooded non-human animal that could benefit from application of the therapeutic packs described herein. While any domestic or feral warm-blooded non-human animal can benefit from the therapeutic packs, the exemplary animals that will benefit from the therapeutic packs will be a feline or canine animal.

As used herein, the term “closure” refers to any means for attaching the therapy packs described herein to the user. Examples of closures include without limitation, hook and loop closures, buckle closures, strap closures, and adhesive closures.

As used herein, the term “closure set” refers to a closure that includes two parts, such as a hook and loop closure where one part of the closure set is the hook part and the other is the loop part. Similar types of closures sets are buckle sets (including quick-release buckle sets), fstrap sets (including cinch strap sets), and adhesive sets.

As used herein, the term “void” refers to any part of the hot or cold therapeutic packs described herein that does not contain the filling material that comprises the claimed therapeutic packs. The void is a distinctly delineated part of the therapeutic pack that is free of the filling material and that acts as a seam so that the therapeutic pack may bend and conform to the contours of the human or animal body parts for which the therapeutic pack is intended.

As used herein, the term “non-linear void” refers to any therapeutic pack void that does not include the filling material and includes any void that is not configured in a straight line, including curved voids, circular voids (e.g., round or oval), or voids having an enclosed non-circular shape (e.g., a square, rectangular, oblong, or heart-shape).

In one embodiment, therapeutic pack is a cold and/or heat therapeutic gel pack. In such a cold or heat gel pack, the filling material will be a gel as described herein and the pack will be reusable via freezing or cooling for the cold gel pack and heating or warming for the heat gel pack. Various different cold and/or heat gel packs for various human and animal body parts are described herein.

In another embodiment, the therapeutic gel pack has a shell material made from a synthetic thermoplastic polymer that is pliable and durable. Examples of such materials include, without limitation, polypropylene, polyvinyl chloride, polyethylene, polycarbonate, polytetrafluoroethylene, nylon, acrylonitrile butadiene styrene, and combinations thereof. In another embodiment, the shell material is transparent.

In a further embodiment, the therapeutic pack is a therapeutic gel pack filled with a thickening agent, water, and an anti-freezing agent. The combination of the thickening agent and the water forms a gel, which provides the gel pack with a degree of structure when used for cold or heat therapy. When the gel pack is used for cold therapy, the anti-freeze agent, which is a liquid that is miscible with water, lowers the freezing point of the water within the gel pack, which allows contents of the gel pack (i.e., the gel formed by the thickening agent and water) to remain in a semi-solid state, i.e., as a gel, below the 0° C. freezing temperature of the water. The presence of the anti-freeze also prevents ice crystals from forming within the gel pack and keeps the pack pliable when cold. When the gel pack is used for heat therapy, the anti-freeze agent will cause the contents of the gel-pack to change from a semi-solid to a liquid state thus allowing more effective heat transfer when applied to a targeted area.

Examples of thickening agents that may be used in the gel pack filling include carboxymethyl cellulose, hydroxyethyl cellulose, sodium polyacrylate silica gel, and combinations thereof. In one embodiment, the thickening agent is carboxymethyl cellulose. In another embodiment, the thickening agent is hydroxymethyl cellulose. In a further embodiment, the thickening agent is sodium polyacrylate. In another embodiment, the thickening agent is silica gel.

Examples of anti-freezing agents that may be used in the gel pack filling include diols and glycols. In one embodiment, the anti-freezing agent is selected from tri-methylene glycol (melting point MP −32° C.; boiling point BP 214° C.), diethylene glycol (freezing point FP −6.5° C.; 244.5° C.), ethylene glycol (FP −12.9° C.; 197.3° C.), propylene glycol (FP −59° C.; BP 188.2° C.), butanediol, glycerol (MP 17.8° C.; BP 290° C.), and combinations thereof. In another embodiment, the butanediol is 1,3-butanediol (butylene glycol) and/or 2,3-butanediol. In a further embodiment, the anti-freezing agent is tri-methylene glycol. In another embodiment, the anti-freezing agent is diethylene glycol. In a further embodiment, the anti-freezing agent is 1,3-butanediol. In another embodiment, the anti-freezing agent is 2,3-butanediol.

In another embodiment, the gel pack comprises a transparent shell of polyvinyl chloride and a filling of water, carboxymethyl cellulose, and tri-methylene glycol.

In a further embodiment, the filling of the therapeutic gel pack comprises a natural food coloring, which provides color to the therapeutic gel pack when the shell material is transparent. Examples of natural food colorings include, without limitation, vegetable sources, such as beet (red), pomegranate (red), raspberry (pink), carrot (orange), paprika (orange), turmeric (yellow), saffron (yellow), chlorophyll (green), matcha (green), spinach (green), parsley (green), purple cabbage (blue), spirulina (blue), blueberry (purple), purple carrots (purple), ube (purple), acai (purple), coffee (brown), cocoa (brown), cinnamon (brown), activated charcoal (black), squid ink (black), and black cocoa (black).

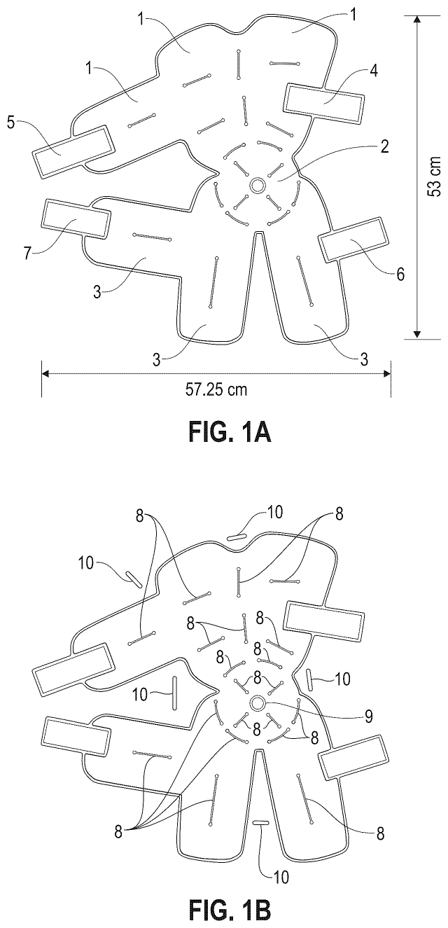

FIGS. 1-29 show a variety of cold and heat therapy gel packs for application to various human body parts.

FIGS. 1A and 1B are schematic diagrams of a half-leg gel pack for application to an adult human leg. With reference to FIG. 1A, the half-leg gel pack wraps around the lower thigh 1, the knee 2, and the calves below the knee 3. The gel pack has a width of 53 cm and a length of 57.25 cm; however, it is to be understood that the gel pack can be manufactured to have different dimensions to suit the knee/leg sizes of various size individuals. With continued reference to FIG. 1A, the closures (one example being a hook and loop, such as VELCRO®, Velcro IP Holdings LLC, Manchester, NH, USA) are used to wrap the half-leg gel pack to the leg of the user. The schematic shown in FIG. 1A has closures that are 12 inches long on the top thigh part of the half-leg gel pack 4, 5 and 10 inches long on the bottom calf part the half-leg gel pack 6, 7; however, it is to be understood that these dimensions are exemplary and that a gel pack can be designed with different dimensions to provide a more secure fit to a user. Where the closures are hook and loop closures, one of the 12-inch closures is the loop closure 4 (the soft side of the hook and loop closure) and the other side of the 12-inch hook and loop closure is the hook closure 5 (the rough side of the hook and loop closure). The 10-inch hook and loop closures on the calf part of the half-leg gel pack have the same arrangement where one side of the 10-inch hook and loop closures is the hook closure 6 and the other side of the 10-inch hook and loop closure is the loop closure 7. With reference to FIG. 1B, the half-leg gel pack is designed with thin void areas 8 that have no gel pack filling; these void areas allow all of the gel packs described herein to have flexibility to conform to the anatomical body part for which the gel pack is intended and to contain the gel-pack within a localized area for maximum therapeutic effect. For additional flexibility at the knee, the knee area of the half-leg gel pack includes a round void area 9 that is placed directly on the kneecap or patella. The shape of the half-leg gel pack also includes empty spaces 10 between the gel pack areas for additional flexibility.

The gel packs described herein are very thin measuring at approximately 1-2 mm without the gel filling material and approximately 5-10 mm with the gel filling material. In one embodiment, the gel packs are approximately 1 mm without the gel filling and approximately 5 mm with the gel filling. In another embodiment, the gel packs are approximately 2 mm without the gel filling and approximately 7 mm with the gel filling. In a further embodiment, the gel packs are approximately 2 mm without the gel filling and approximately 10 mm with the gel filling.

The dimensions of the voids, which along with the very thin overall dimensions of the gel pack, allow the gel pack to conform anatomically to the body part for which the gel packs are intended. In order to conform to anatomical body parts that curve, flex, and/or bend, the gel packs may require a plurality of voids, which may be linear voids, non-linear voids, or a combination of linear and non-linear voids. The non-linear voids may be curved, arched, circular, oval, or any other shape required to allow the gel pack to bend and flex according to the body part that it is intended to cover. The dimensions of the voids will vary depending on the anatomical design of the gel packs. Where the voids are linear, the width of a typical void will generally be in the range of 1-5 mm wide and the length of a typical void will vary from 2-10 cm depending on the size and configuration of the particular gel pack. In one embodiment, the void is approximately 1 mm wide and 2 cm in length. In another embodiment, the void is approximately 1 mm wide and approximately 4 cm in length. In a further embodiment, the void is approximately 2 mm wide and approximately 7 cm in length. In another embodiment, the void is approximately 2 mm wide and approximately 10 cm in length. In a further embodiment, the void is approximately 3 mm wide and approximately 10 cm in length. In another embodiment, the void is approximately 4 mm wide and approximately 10 cm in length. In a further embodiment, the void is approximately 5 mm wide and approximately 10 cm in length. Where the voids are non-linear, the width of a typical void will also be 1-5 mm wide. The dimensions of all voids will depend on the dimensions of the body part to which the gel pack is to be applied. For example, with reference to FIG. 1C, the circular patella void may have a diameter in the range of 3-6 cm.

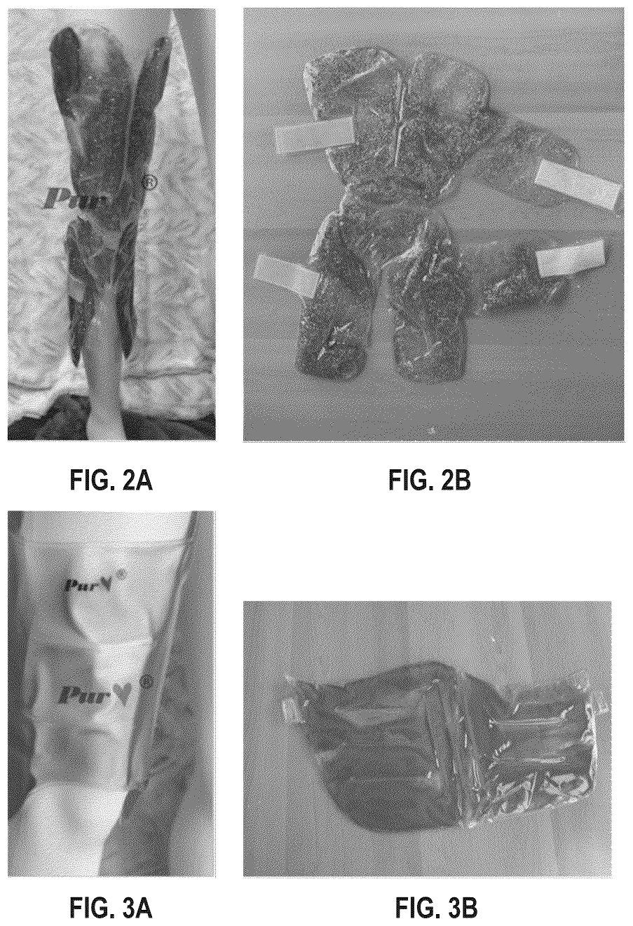

FIGS. 2-11 show gel packs designed for application to the legs, ankles, and feet of a human user.

FIG. 2A shows the half-leg gel pack as described herein and as shown schematically in FIGS. 1A and 1B as applied to a mannequin leg. As shown therein, the half-leg gel pack covers a good portion of the mannequin's lower thigh and upper calf as well as completely covering the mannequin's knee. As shown in FIG. 2B, this gel pack is secured with two sets of closures, one set of closures that attach around the leg at the lower thigh above the knee and the other set of closures that attach at the upper calf below the knee. The circular area of the gel pack between the upper and lower portions of the gel pack allows the gel pack to bend comfortably at the knee.

FIGS. 3A and 3B show a gel pack for application to the upper leg of a user. This gel pack is secured with a closure set that attaches at the back of the leg at the lower thigh above the knee. Applications for such upper leg gel pack include, without limitation, thighplasty, transverse media thigh lift, and L-shaped inner thigh lift.

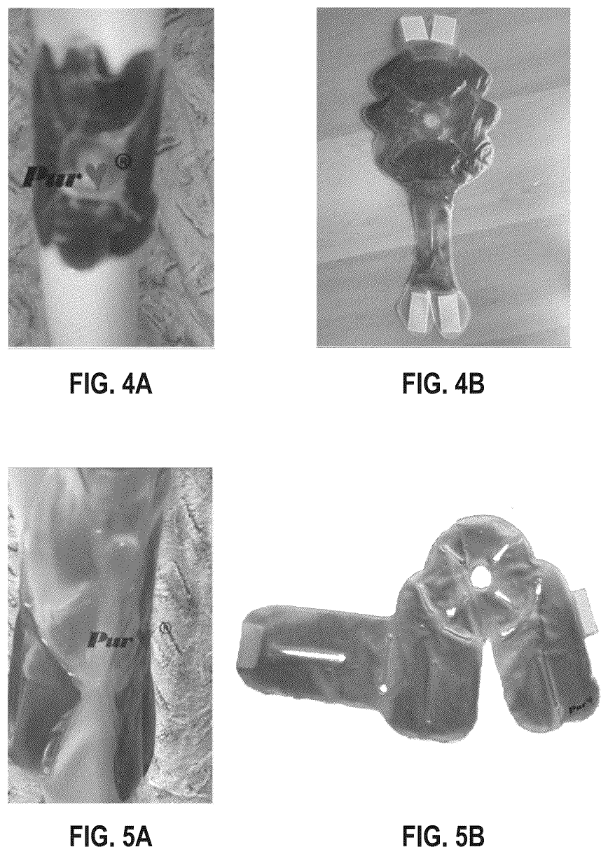

FIGS. 4A, 4B, 5A, and 5B show gel packs for application to the knee of a user. The gel pack of FIGS. 4A and 4B covers just the knee area while the gel pack of FIGS. 5A and 5B covers the knee and a portion of the leg just below the knee. The knee gel packs are secured with one or two sets of closures (FIG. 4B shows two sets of closures set closely together and FIG. 5B shows a single set of closures) that attach at the side of the knee. Both knee gel packs have a single linear void at the back of the knee for flexibility. Applications for such knee gel packs include, without limitation, ACL reconstruction surgery, knee arthroplasty, torn posterior cruciate ligament (PCL), knee replacement surgery, knee osteotomy, and cartilage restoration.

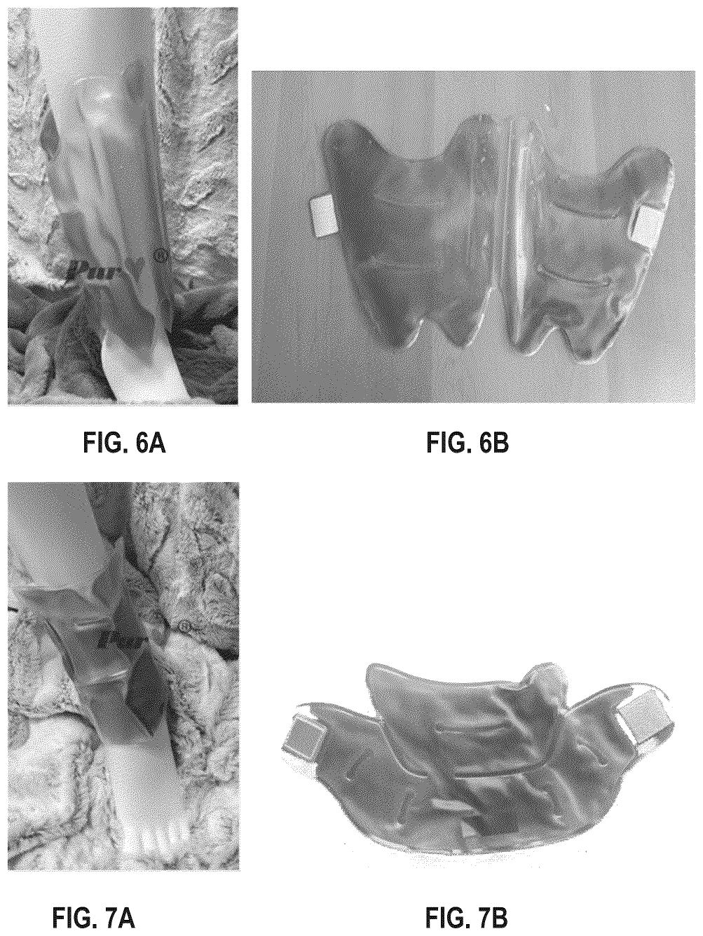

FIGS. 6A and 6B show a gel pack for application to the lower leg of a user. This gel pack is secured with a closure set that attaches at the lower calf. Applications for such a lower leg gel pack include, without limitation, calf injuries and calf augmentation surgery.

FIGS. 7A and 7B show a gel pack for application to the ankle of a user. This gel pack is secured with a closure set that attaches at the ankle. Applications for such an ankle gel pack include, without limitation, Achilles rupture, ankle impingement, peroneal tendinopathy, syndesmotic injuries, and clubfoot corrections.

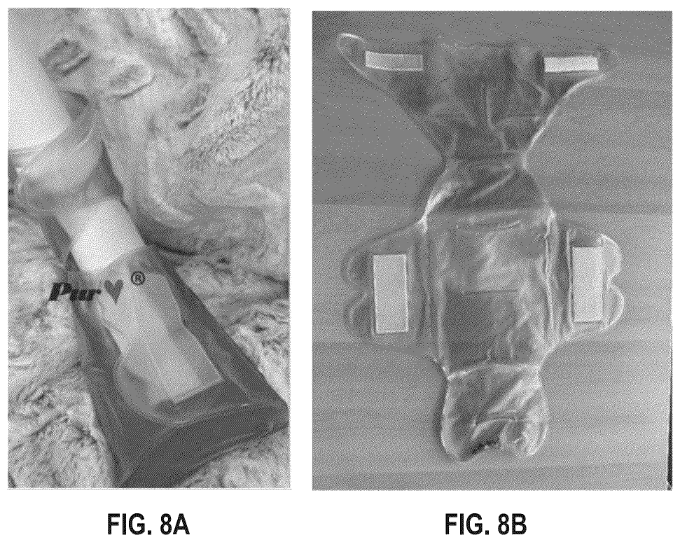

FIGS. 8A and 8B show a gel pack for application to the whole foot of a user. This gel pack covers the toes, upper foot, sole of the foot, and ankle of the user. This gel pack is secured in place with two sets of closures, one set that attaches at the top of the foot of the user and the other set that attaches at the ankle. Applications for such whole foot gel pack include, without limitation, fusion surgery, hallux rigidus, plantar fasciitis, metatarsal surgery, Morton's neuroma, and foot fractures.

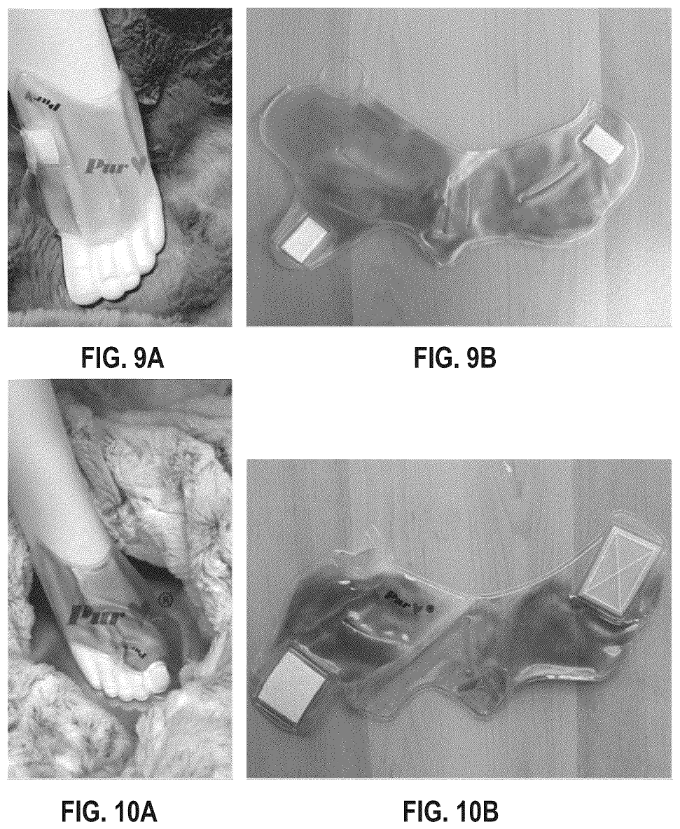

FIGS. 9A and 9B shows a gel pack for application to half of the foot of a user. This gel pack includes a toe loop and surrounds the foot of the user, but does not cover the toes or ankle. This gel pack is secured with the toe loop and a closure set that attaches at the side of the foot at or around the arch area. Applications for such half foot gel packs include, without limitation, hallux rigidus and wedge osteotomy.

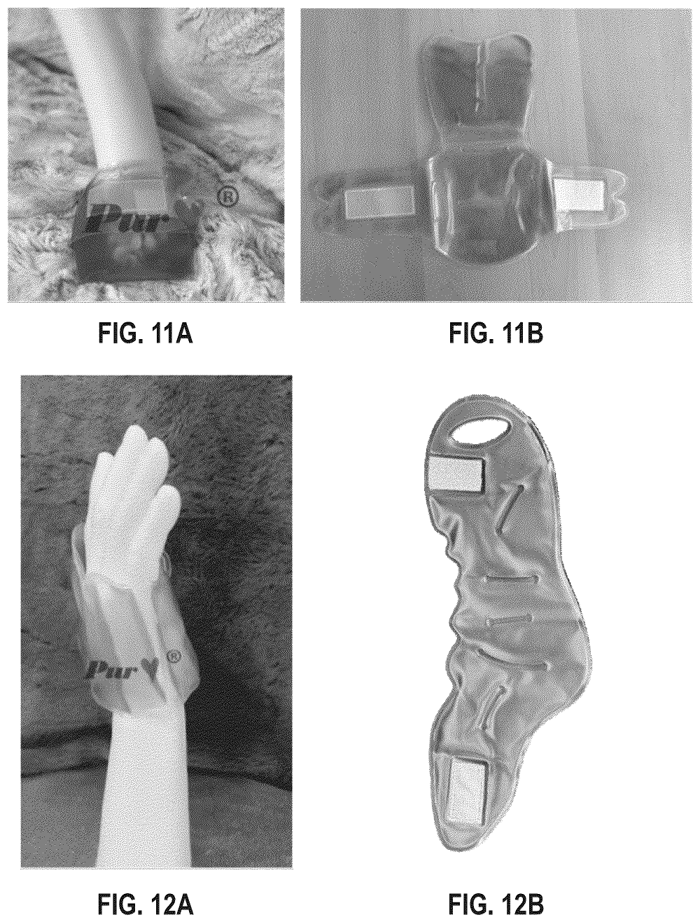

FIGS. 10A and 10B show a gel pack for application to the metatarsophalangeal (MTP) joint area at the base of the big toe of the user. This gel pack includes a toe loop and surrounds the lower part of the foot including the MTP joint. This gel pack is secured with the toe loop and a closure set that attaches at the bottom of the foot below the MTP joint. Application for such a lower foot gel pack includes, without limitation, bunionectomy.

FIGS. 11A and 11B show a gel pack for application to the toes of the foot of a user. This gel pack is designed with two voids that allow the gel pack to bend over the toes. This gel pack is secured with a closure set that can be attached at the top or the bottom of the foot. (FIG. 11A shows the gel pack secured at the top of the foot). Applications for such a toe gel pack include, without limitation, fusion surgery, hallux rigidus, and toe deformity corrections.

FIGS. 12-17 show gel packs for application to the arms and hands of a human user.

FIGS. 12A and 12B shows a gel pack for application to the hand of a user where the gel pack has an opening for the thumb and wraps around the hand to cover the top of the hand and the palm. This gel pack is secured to the hand by the thumb hole opening and a closure set that attaches just below the thumb. This gel pack does not cover the wrist of the user. Applications for such hand gel packs include, without limitation, Dupuytren's contracture, de Quervain's tendinosis surgery, joint replacement, tendon repair, and nerve repair.

FIGS. 13A and 13B shows a gel pack that covers the palm, but not the top of the hand. This gel pack is secured with two loops that go around the thumb and a closure set that secures at the wrist on the top part of the hand/wrist area. Applications for such hand gel packs include, without limitation, Dupuytren's contracture, de Quervain's tendinosis surgery, joint replacement, tendon repair, nerve repair, and carpel tunnel syndrome.

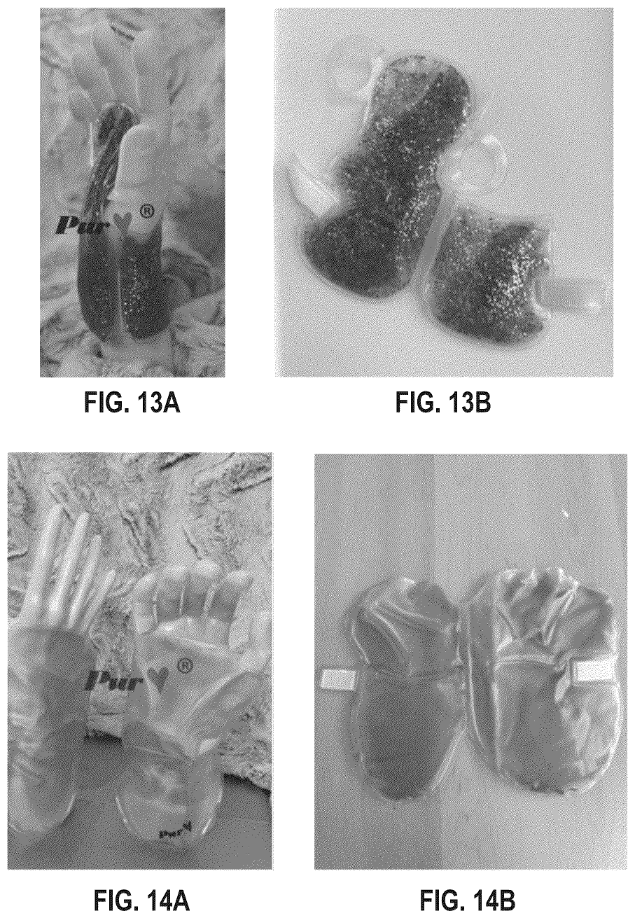

FIGS. 14A and 14B show a gel pack for application to the hand and wrist of a user. This gel pack is secured with a closure that goes around the lower part of the hand or wrist area of the user. Application for such a hand/wrist gel pack includes, without limitation, arthritis and carpal tunnel syndrome.

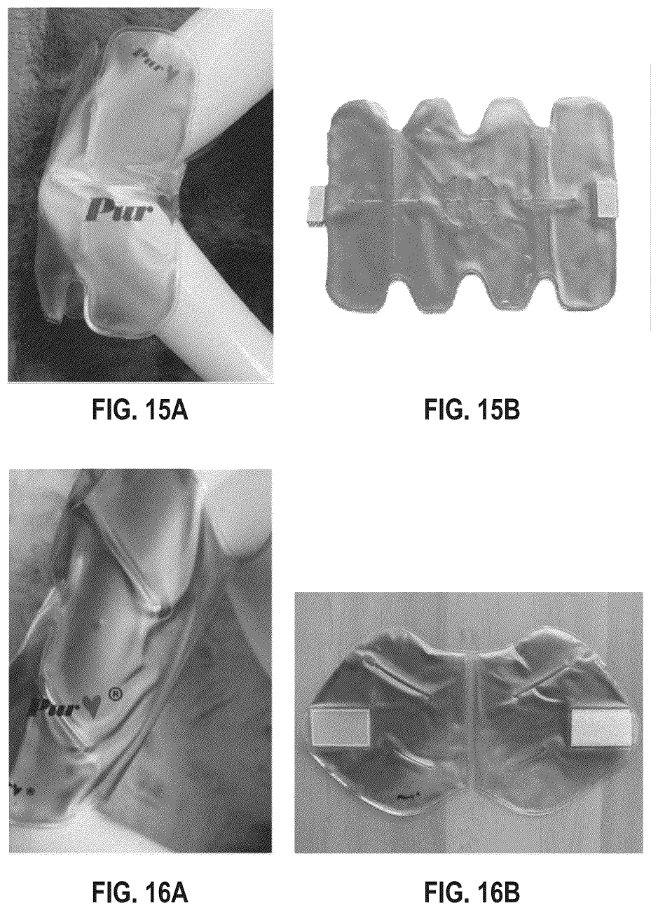

FIGS. 15A and 15B show a gel pack for application to the elbow of a user. This gel pack is secured Application for such an elbow gel pack includes, without limitation, tennis elbow surgery, ulnar collateral ligament reconstruction (UCL or Tommy John surgery), arthroscopy, brachioplasty, and elbow tuck.

FIGS. 16A and 16B shows a gel packs for application to the upper arm of a user. This gel pack is secured to the upper arm of the user with a single set of closures that connects at the back of the upper arm of the user. Applications for such upper arm gel packs include, without limitation, upper arm lift and cutaneous lymphoma.

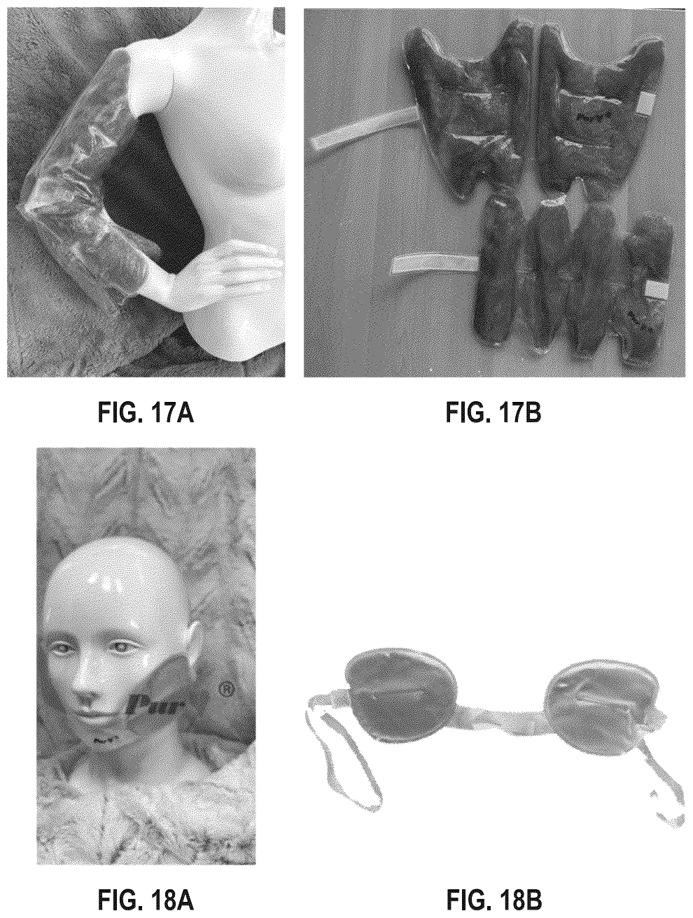

FIGS. 17A and 17B show a gel pack for application to the entire arm of a user. As shown in FIG. 17B, this gel pack is secured in two places with one set of closures securing the gel pack at the upper arm and the second set of closures securing the gel pack at the lower arm Applications for such a full arm gel pack include any type of full arm injury, such as strains, fractures, and dislocations.

FIGS. 18-20 show gel packs for application to the head, face, torso, and trunk of a human user.

FIGS. 18A and 18B show a gel pack for application to the cheeks of a user. The cheek gel pack includes two round gel packs connected together with a flexible ribbon-like attachment that is placed below the mouth on the chin of the user and the cheek gel pack is secured with a closure that attaches at the back of the head. In FIGS. 17A and 17B, the two round gel packs have a single void that allows the gel packs to conform to the contours of the user's cheek. Application for such a cheek gel pack includes, without limitation, cheek implant surgery and buccal fat removal surgery.

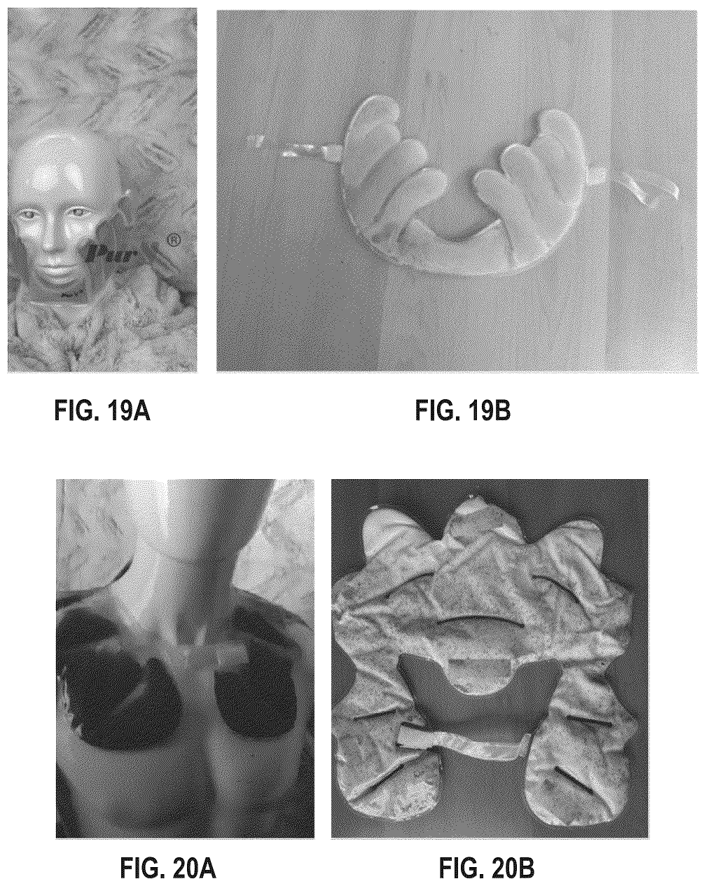

FIGS. 19A and 19B show a gel pack for application to the lower face of a user. This gel pack is designed to cover the chin and both sides of the face of the user from the mouth to the ears. The gel pack fully covers the cheeks and includes several vertical voids to facilitate the bending of the gel pack around the contours of the lower face. Like the cheek gel pack in FIGS. 19A and 19B, the lower face gel pack is secured to the lower face of the user with a closure that is fastened at the back of the head. Applications for such a lower face gel pack include, without limitation, lower face lift surgery or jowl lift surgery.

FIGS. 20A and 20B show a gel pack for application to the back neck, shoulders, upper back, and upper chest area. As shown in FIG. 20B, the gel pack includes three non-linear voids at the back neck and upper back and four linear voids at the shoulders and upper chest, all of which combined, allow the gel pack to drape over the shoulders of the user and conform to the target areas of the gel pack (i.e., the back neck, shoulders, upper back, and upper chest). The gel pack is secured at the front of the gel pack below the neck at the collarbone with two closures, which allow the user to ensure a proper fit of the gel pack. Applications for such a gel pack include, without limitation, neck and shoulder injuries or pain and heat exhaustion.

In another embodiment, the gel pack does not include a closure set, but instead, is attached to the user with a self-adhesive bandage, which is wrapped around the gel pack until it secured at its point of application on the user.

The gel packs described herein with the hook and loop closures may be stored long term in a freezer. When properly cared for, the gel pack should last for several years and/or thousands of uses.

In one aspect, the gel pack is used for cold therapy. The active cold time for a frozen gel pack as described herein will be approximately 20-40 minutes at which time, the gel pack may need to be placed back into the freezer. The contents of a gel pack at room temperature will take approximately one hour to freeze.

In another aspect, the gel pack is used for heat therapy. A frozen or room temperature gel pack can be heated with a water bath or through microwaving. When heating with a water bath, a container of water should be brought to a boil and removed from the heat prior to the immersion of the gel pack into the container of water. Upon immersion, the gel pack should be removed after 4 minutes for a temperature check. If more heat is required, the gel pack should be re-immersed into the water bath for additional 1-minute intervals with temperature checks carried out after each re-immersion until the desired temperature is reached. When heating with a microwave, times will vary depending on the power of the microwave. For a conventional 600 W microwave, a frozen gel pack is placed into the microwave and heated on high power for approximately 3 minutes. If a gel pack is already at room temperature, 1 minute of microwave time is all that is required. If additional heat is needed, additional microwaving should be carried out in 10-second intervals until the desired temperature is reached.

In a further aspect, the cold therapy gel pack described herein is used for acute injuries or for chronic inflammation. In application, a cold therapy gel pack is applied directly to the injured area for 15-20 minutes at a time and repeated as necessary.

In another aspect, the heat therapy gel pack described herein is used to loosen muscles and joints and to relax injured tissue before activities. In application, a heat therapy gel pack is applied directly to the injured joint or muscle for 20 minutes at a time and repeated as necessary.

In another embodiment, the gel packs described herein may be attached to the skin of the user with a medical adhesive instead of hook and loop closures, where the medical adhesive is applied to one side of the gel packs. The medical adhesives used for the gel packs will be medical grade pressure sensitive adhesives, such as for example, synthetic rubber, acrylic, soft silicone gel, epoxy-polyurethane blends, cyanoacrylates, and combinations thereof. The medical adhesive gel packs will generally be used for cold therapy since heating can damage the chemical properties of adhesives, making them structurally weak. High temperatures can also cause pressure sensitive adhesives to melt and release an odor.

In a further embodiment, the medical adhesive is in the form of a medical tape. In another embodiment, the medical tape is pressure sensitive tape comprising polyethylene terephthalate and medical grade acrylic adhesive. In one embodiment, the medical tape is a double-sided medical tape that is attached to one side of the gel pack where the other side of the medical grade adhesive is covered with an adhesive backing until the gel pack is ready for use at which time the adhesive backing is removed and the gel pack is applied to the skin of the user at the site requiring therapy. In another embodiment, one side of the gel pack is manufactured directly with the medical tape, which is covered with an adhesive backing until the gel pack is ready for use. In a further embodiment, upon removal, the adhesive backing of the medical adhesive gel packs may be stored for reuse of the gel packs. If the adhesive backing of the medical adhesive gel packs is properly stored, it may be reapplied to the gel packs immediately after use. With proper care, storage, and use, the medical adhesive gel packs may be reused up to 100 times.

In a further embodiment, the medical adhesive of the gel packs may contain a therapeutic agent. Examples of therapeutic agents that may be added to the adhesive include, without limitation, antibiotic agents, anti-inflammatory agents, pain relieving agents, anesthetics, and combinations thereof. In one embodiment, the medical adhesive contains Arnica montana, which is a therapeutic agent with anti-inflammatory and pain-relieving properties. In another embodiment, the medical adhesive contains a first aid type antiobiotic agent, such as for example, neomycin, bacitracin, polymyxin, and combinations thereof.

In another embodiment, the gel packs described herein may include one or more sterile dressing pads embedded within the gel pack. The dressing pad gel packs are intended for single use applications as the dressing pads will no longer be sterile after use. The one or more sterile dressing pads may be embedded within any of the gel packs described herein while being tailored for the application of the particular gel pack. For example, if a dressing pad gel pack is needed for heat therapy, then the dressing pad will be embedded in a gel pack that has a closure without any metal (such as for example, a gel pack with a hook and loop closure) or a gel pack without a closure, both of which will allow for the gel pack to be subject to heating. By contrast, if the dressing pad gel pack is needed for cold therapy, then the dressing pad may be embedded in a gel pack with any type of closure, a medical adhesive backed gel pack, or a gel pack without a closure (which may secured to the user with a self-adhering bandage), all of which may be placed in a freezer to prepare the gel packs for cold therapy. Where close contact of the dressing pad with the skin is required, the dressing pad gel packs may be most effective when embedded within a medical adhesive gel pack.

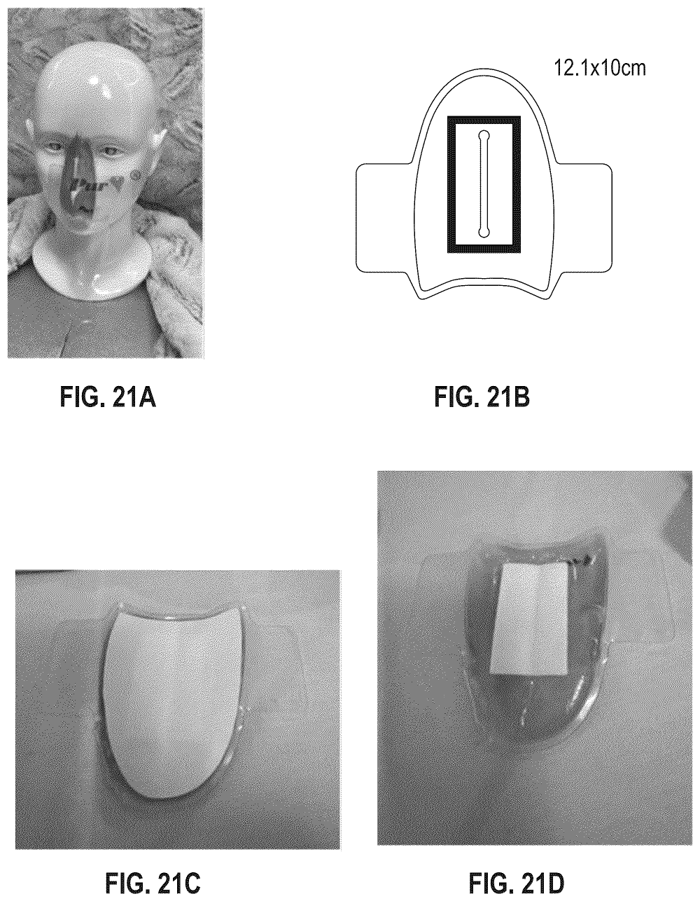

FIGS. 21-29 show exemplary single use medical adhesive gel packs with and without dressing pads.

FIGS. 21-25 show exemplary medical adhesive dressing pad gel packs for application to the face, neck, and spine of a user.

FIGS. 21A-21D show a medical adhesive dressing pad gel pack with an oval shape that is concave at one end and convex at the other end that may be used where such a shape is desirable. FIG. 21A shows application of the gel pack to the nose of a mannequin. Examples of applications where a nose gel pack could be used include, without limitation, septoplasty, rhinoplasty, and nasal valve repair. FIG. 21B is a diagram of the gel pack showing the position of the dressing pad on the void area of the gel pack. FIG. 21C shows the gel pack with the protective adhesive backing for the dressing pad and FIG. 21D shows the gel pack with the protective adhesive backing removed showing the dressing pad that is embedded in the gel pack. Prior to its removal, the adhesive backing maintains the sterile state of the dressing pad. Upon removal, the dressing pad and adhesive on the gel pack is exposed. The adhesive that will allow the gel pack to adhere to the user is on the gel pack surface and not on the dressing pad. The transparent wings on the side of the gel packs allow the user to apply the gel pack without the risk of touching the adhesive or the sterile dressing pad. The thin void area 8 shown in FIG. 21C provides the gel pack with flexibility as described in the discussion of FIG. 1B.

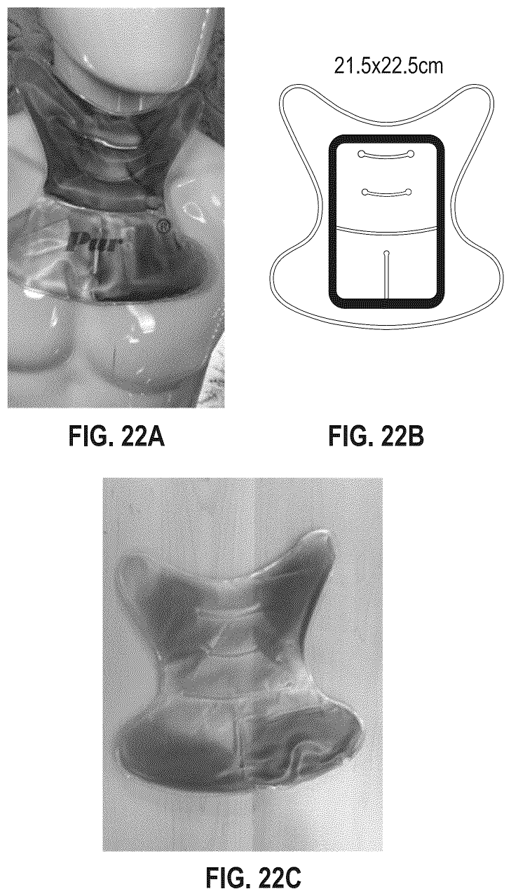

FIGS. 22A-22C show a medical adhesive dressing pad gel pack for application to the neck and throat of a user. Application for such a dressing pad gel pack includes, without limitation, cervical disc surgery and thyroid biopsy.

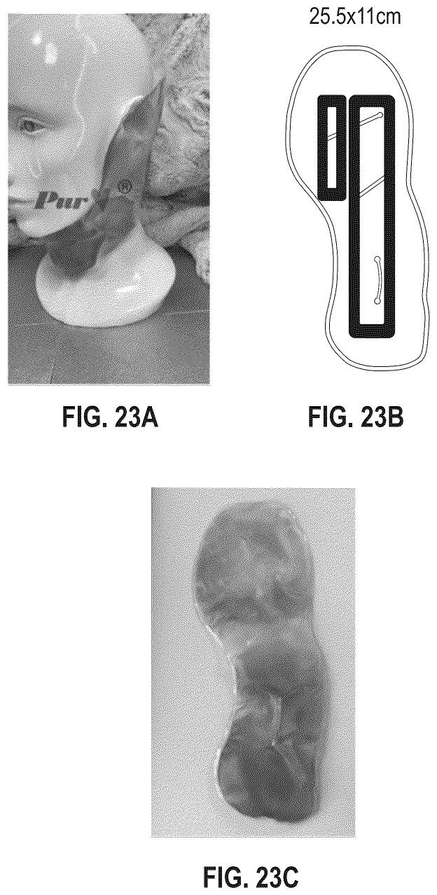

FIGS. 23A-23C show a medical adhesive dressing pad gel pack for application to the neck and ear of a user. Application for such a dressing pad gel pack includes, without limitation, parotid surgery.

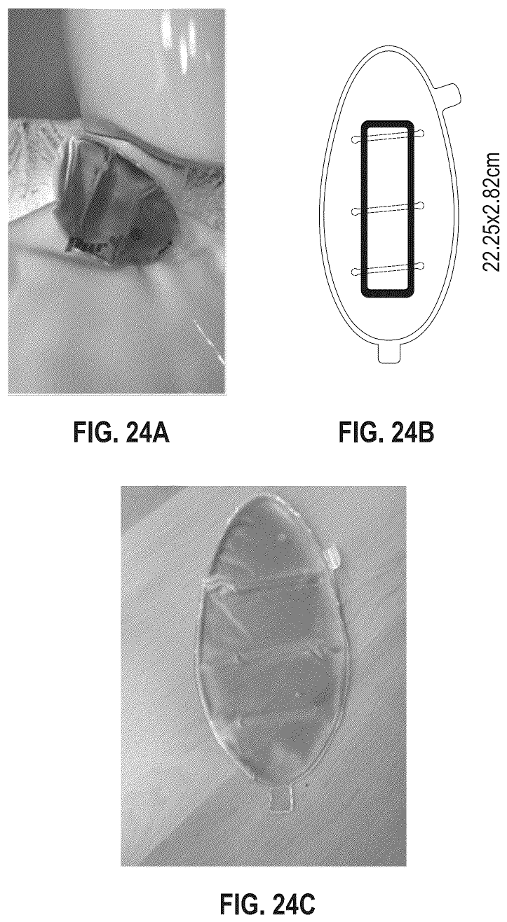

FIGS. 24A-24C show a medical adhesive dressing pad gel pack for application to the neck of a user. Application for such a dressing pad gel pack includes, without limitation, carotid surgery.

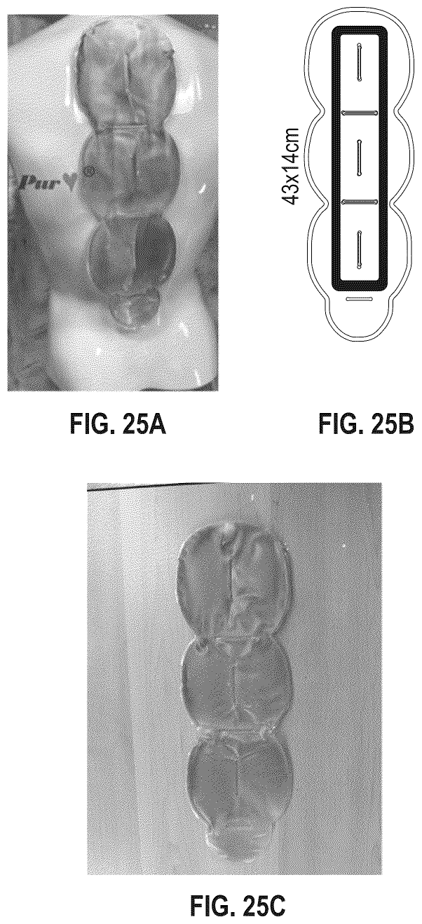

FIGS. 25A-25C show a medical adhesive dressing pad gel pack for application to the spine and back of a user. Application for such a dressing pad gel pack includes, without limitation, herniated disc surgery, laminectomy, discectomy, spine osteotomy, vertebroplasty, kyphoplasty, and bone marrow aspiration.

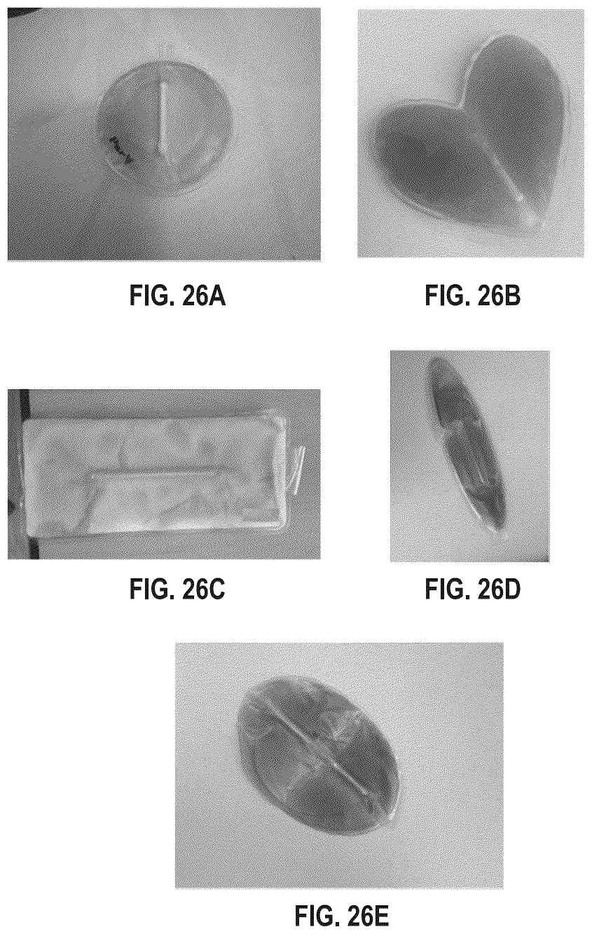

FIGS. 26A-26E show various different shapes of the medical adhesive gel packs as described herein. Each medical adhesive gel pack will include a void for flexibility as described herein and may include a dressing pad if desired. The gel packs may also include an extra notch without adhesive for ease of application to the body of the user. Such medical adhesive gel packs may have applications as cold therapy gel packs where such shapes are suitable, such as for example, the cheeks, shoulders, back, chest, arms, and legs.

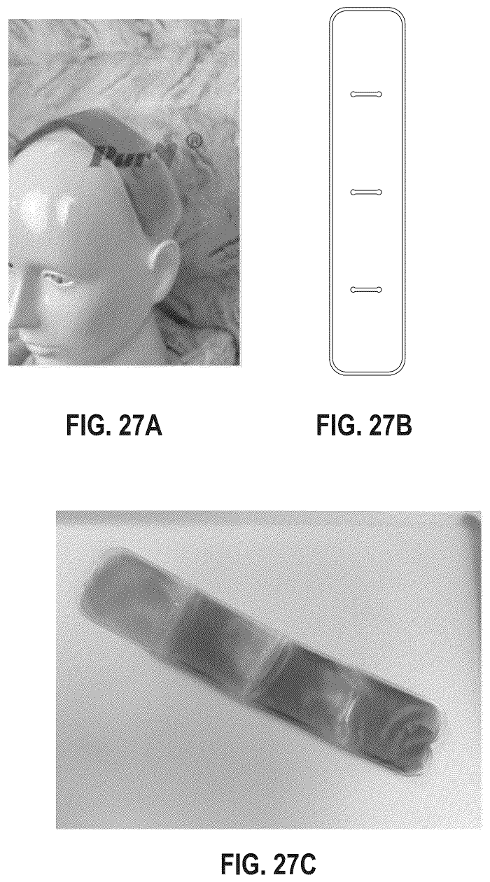

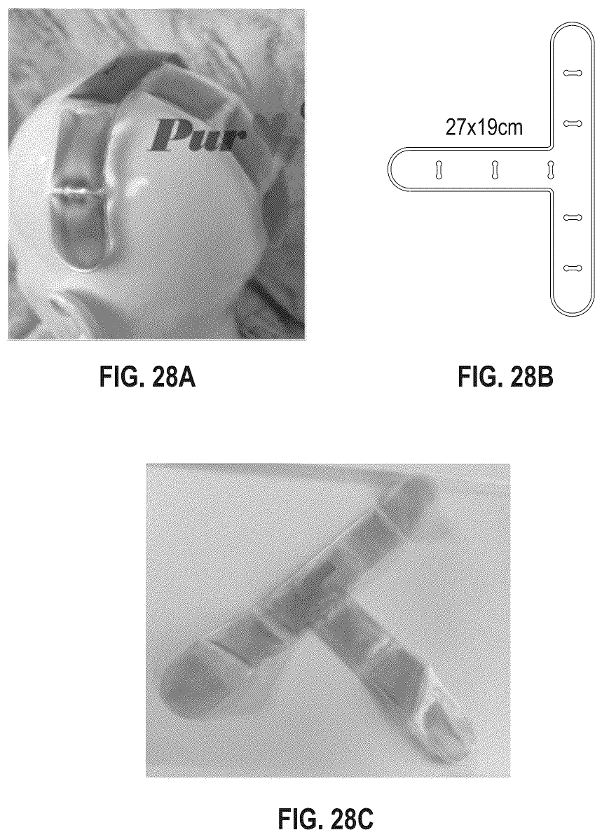

FIGS. 27A-27C and FIGS. 28A-28C show medical adhesive gel packs applied to scalp/head of a user. To conform to the head of the user, such gel packs will include multiple voids allow for the gel pack to bend over the curves of the scalp. For such scalp/head gel pack, the medical adhesive will preferably be applied to the most terminal segments of the gel pack. Applications for such a medical adhesive gel pack include, without limitation, lesionectomy, corpus callosotomy, functional hemispherectomy, and seizures.

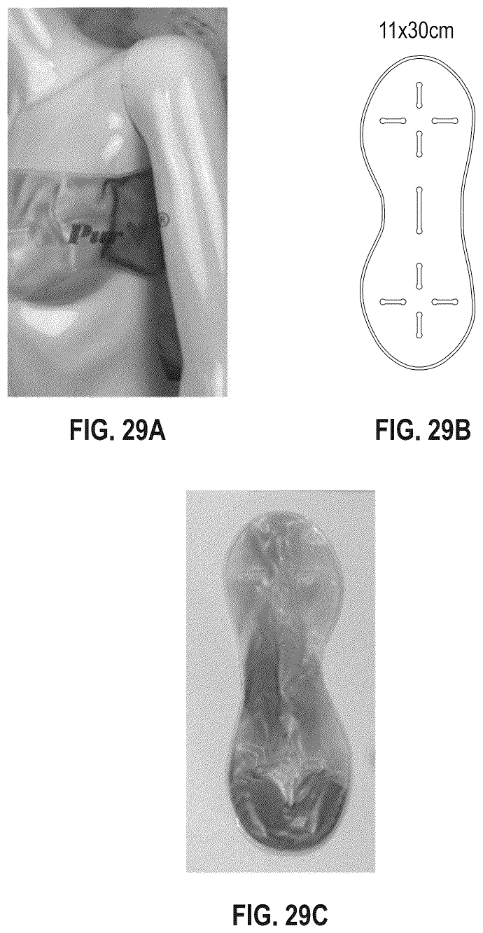

FIGS. 29A-29C show a medical adhesive gel pack for application to the breast and underarm of a user. Application for such a medical adhesive gel pack includes, without limitation, mastectomy and lumpectomy. The medical adhesive gel pack of FIGS. 29A-29C may be equipped with a dressing pad if desired.

In a further embodiment, the cold or heat therapy gel packs described herein may be placed inside a compression garment that wraps around a body part of the user. In such an embodiment, the therapy gel pack will not require a means for attaching, but rather will be a stand-alone gel pack of a size that will accommodate the pocket of the compression garment.

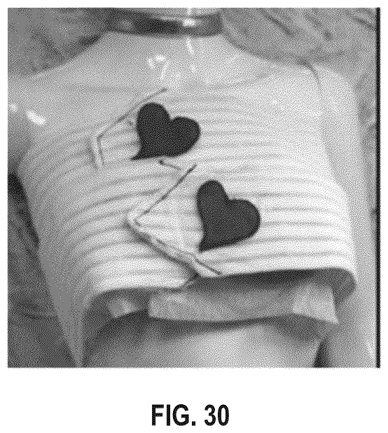

FIG. 30 shows an exemplary compression garment designed to wrap around the chest of a user, wherein the compression garment contains pockets to contain one or more of the gel packs described herein. Where the compression garment is used, it is to be understood that the gel packs that are inserted into the compression garment will not include a medical adhesive.

While a dressing pad gel pack may be used, it is not necessary as the compression garment, rather than the gel pack will be in contact with the skin of the user. To conform to the contours of the chest of the user, the gel packs within the pockets of the compression garment will contain the voids that allow the gel packs to conform closely to the anatomical contours of the user's chest.

In a further embodiment, any of the gel packs described herein may be embedded with a medical grade silicone tape sheet instead of a dressing pad. Medical grade silicone tape is a safe and effective tape for scar management. The silicone tape may be embedded in the single use medical adhesive gel packs or in the multiple use closure set gel packs. As medical grade silicone tape may reused and may withstand some degree of heating, gel packs equipped with closures sets and medical grade silicone tape may be used for both cold and heat therapy and may be washed with water and reused for a period of time.

Medical grade silicone tape is a thin, flexible, and breathable self-adhesive tape that is designed to soften and flatten scars, reduce scar redness, alleviate scar-related pain and itching, and to reduce the overall appearance of scars. Silicone tape is effective for application to new or old scars from burns, surgeries, and other trauma. By creating a moist and occlusive barrier, silicone tape helps to hydrate the scar tissue, normalize the skin's barrier function, and reduce excessive collagen production, which leads to a more normal looking, less red, and less raised scar. In application, silicone tape provides hydration, collagen regulation, pressure and tension, and an increase in the temperature of the skin. With regard to hydration, the silicone tape provides a moist environment by preventing water loss from the skin. This hydration helps to restore the stratum corneum, the outermost layer of skin, to a normal state. With regards to collagen regulation, in hypertrophic and keloid scars, the stratum corneum absorbs too much water, leading to excessive collagen production. By normalizing hydration, silicone therapy reduces the stimulus for excessive collagen. With regard to pressure and tension, the flexibility of the silicone tape allows the tape to transfer tension from the wound edges to the sheet of tape, which helps to prevent abnormal scar growth like keloids. With regard to increased temperature, the covered area also experiences a slight temperature increase, which can limit blood flow and reduce the excessive healing processes involved in scarring.

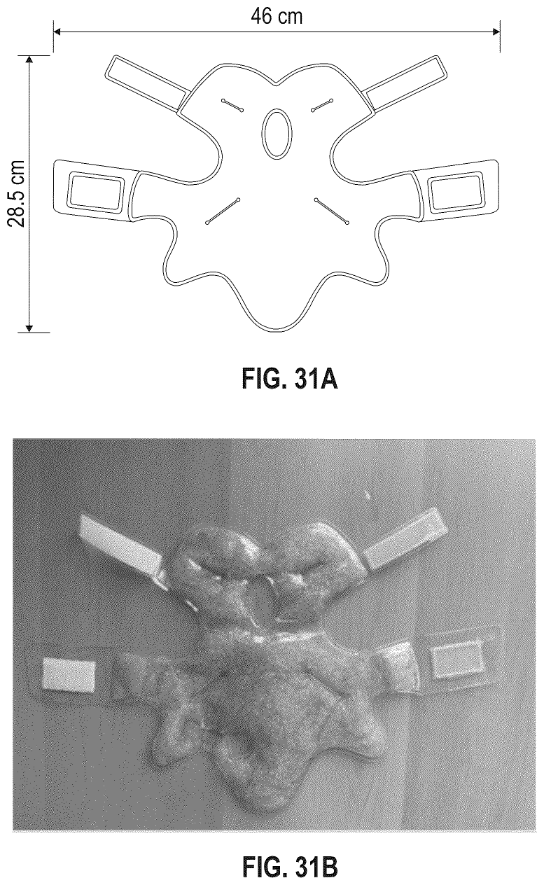

The next set of gel packs described herein are uniquely designed for application to non-human animals. FIGS. 31-36 are schematic diagrams and corresponding photographs of exemplary cooling therapy gel packs for feline and canine animals. The animal gel packs described herein a custom wearable gel packs that are designed to apply cold therapy to the body of the animal by wrapping an area or combination areas of the body, such as the neck, front body, chest, back body, armpit, groin, and/or abdomen. The animal cold therapy gel pack has application as a cooling remedy to relax muscles, reduce anxiety, stimulate endorphins release, aid in pain management, aid in injury healing, aid in recovery following medical procedures and surgeries, prevent heatstroke, and minimize heat exhaustion and panting from heat stress. As is known to those of skill in the art and to owners of feline or canine animals, heatstroke is a severe illness characterized by heating panting, rapid heartbeat, and difficulty breathing. Heat stroke is most common among elderly, young, and overweight animals as well as animals with heart or respiratory diseases. In use, the cooling gel packs may be stored in a freezer, but should be moved to a refrigerator until the gel packs reach a temperature below freezing (i.e., 32 ° F. or 0° C.); the packs should thus be cool to cold, but not extremely cold, to the touch. Like the hot or cold therapy gel packs for application to humans, the cooling gel packs for animals are designed with voids to ensure that the gel packs can conform to the contours of the animal bodies. The cooling gel packs for the animals may also include larger voids to ensure a proper fit of the gel packs.

FIG. 31A is a schematic diagram of a cooling vest for application to the back of a feline or canine animal and FIG. 31B is a photograph of the cooling vest as manufactured according to the schematic diagram. The cooling vest targets the back of the animal (the area of the animal body along the backbone) and may be used to keep animals that struggle with heat or that have thick fur comfortable during hot weather. The schematic diagram at FIG. 31A shows a 46 cm (18.1 in) length×28.5 cm (11.2 in) width×1 cm (0.4 in) height cooling vest with two closures; however, it is to be understood that the cooling vest may be manufactured in a variety of sizes to fit a variety of animals and if required, more closures. The two closures shown in FIGS. 31A and 31B secure the cooling vest to the animal by attaching (via hook and loop, buckle, strap, adhesive, self-adhering bandages etc.) at the front throat area (i.e., the cranial neck area) and at the belly (i.e., the ventral part) of the animal. The four thin corner voids, which may be linear or curved (the voids in FIGS. 31A and 31B are thin linear voids) and the larger oval void in the center of the gel pack allow the vest to be flexible to fit atop the back area of the animal.

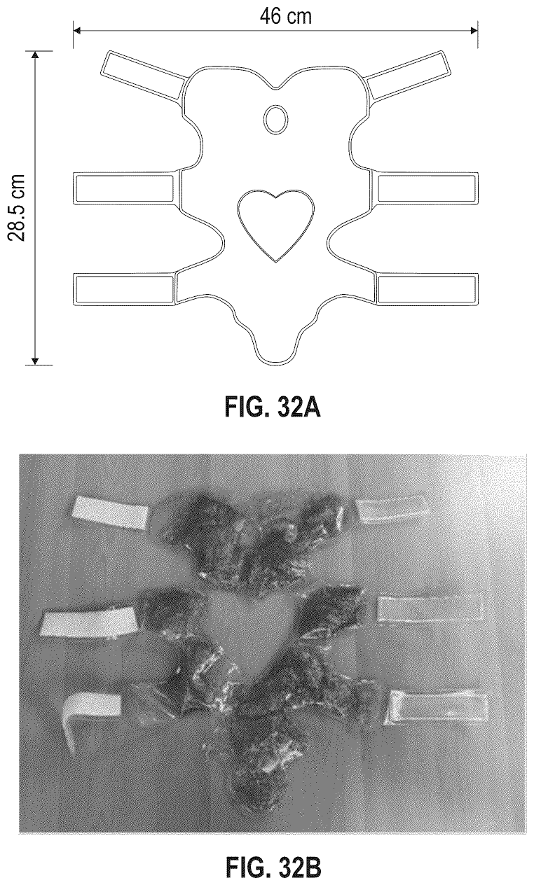

FIG. 32A is a schematic diagram of a cooling suit for application to the front and underside of a feline or canine animal and FIG. 32B is a photograph of the cooling suit gel pack as manufactured according to the schematic diagram. The cooling suit targets the neck, chest, armpits, and groin to help the animals suffering from heatstroke by facilitating rapid cooling of the neck, chest, armpits, and groin. Due to the large area of the animal that is targeted by the cooling suit, it is one of the largest of the gel packs described herein. The schematic diagram at FIG. 32A shows a 63 cm (24.8 in) length×49 cm (19.3 in) width×1 cm (0.4 in) height cooling suit with three closures; however, it is to be understood that the cooling suit may be manufactured in a variety of sizes to fit a variety of animals and if required, more closures. In order to fit around the neck, chest, armpits, and groin of the animal, the first set of closures secure at the back of the neck (i.e., the dorsal neck), the second set of closures secure at the upper back of the animal (i.e., the upper dorsal back), and the third set of closures will secure at the lower back of the animal (i.e., the lower dorsal back). Due to the large size of the cooling vest, a larger void, such as the large heart-shaped void shown in FIGS. 32A and 32B, may provide the cooling vest with greater flexibility than the thin voids that are integrated into the smaller gel packs described herein. Within the context of the present invention, a large void, such as the heart-shaped void shown in FIGS. 32A and 32B is considered a non-linear void.

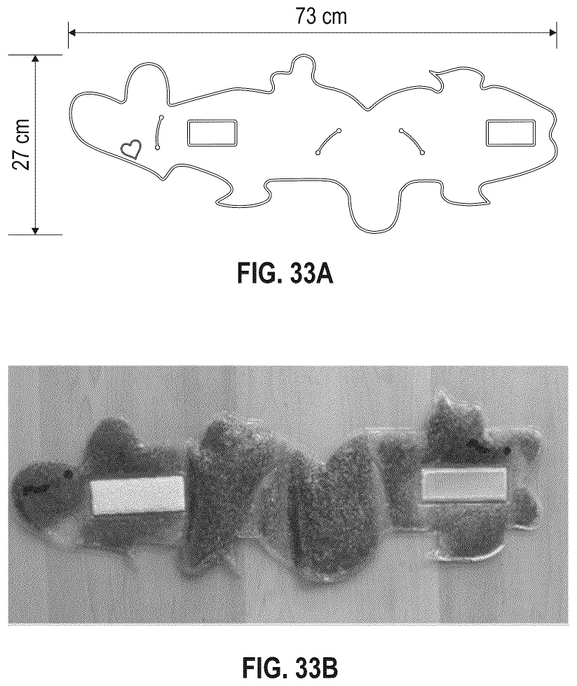

FIG. 33A is a schematic diagram of a cooling belly gel pack for application to the abdomen area of a feline or canine animal and FIG. 33B is a photograph of the cooling belly gel pack as manufactured according to the schematic diagram. The cooling belly gel pack has application as a cold compress following spay or neuter surgeries. Due to the large area that is targeted by the cooling belly gel pack, it is one of the longest gel packs described herein. The schematic diagram at FIG. 33A shows a 73 cm (28.7) length×27 cm (10.6 in) width×1 cm (0.4 in) height cooling belly gel pack with a single closure; however, it is to be understood that the cooling belly gel pack may be manufactured in a variety of sizes to fit a variety of animals and if required, more closures. The single closure secures the cooling belly gel pack to the animal by attaching at the abdomen (i.e., the ventral trunk area) of the animal. The three curved voids are integrated into the cooling belly gel pack to allow the gel pack to be flexible to fit around the abdomen of the animal.

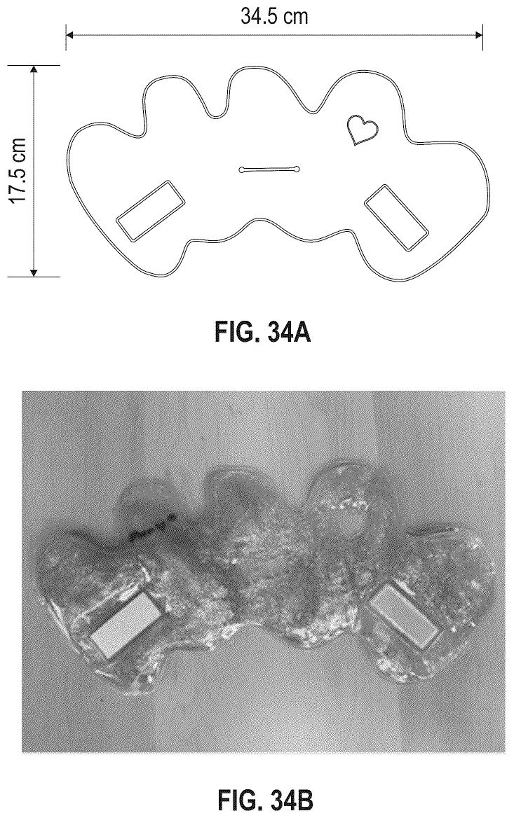

FIG. 34A is a schematic diagram of a collar gel pack for application to the neck of a feline or canine animal and FIG. 34B is a photograph of the collar gel pack as manufactured according to the schematic diagram. The collar gel pack has application following spay or neuter surgeries by forming a barrier around the base of the neck to stop the animal from using their mouth to reach the incision site while simultaneously offering temperature relief without limiting mobility. The schematic diagram at FIG. 34A shows a 34.5 cm (13.6 in) length×17.5 cm (6.9 in) width×1 cm (0.4 in) height collar gel pack with a single closure; however, it is to be understood that the collar gel pack may be manufactured in a variety of sizes to fit a variety of animals. The single linear void is integrated into the collar gel pack to allow the gel pack to be flexible to fit around the neck area of the animal. To apply the collar gel pack to the animal, the gel pack is first folded and then the folded gel pack is fitted around the neck of the animal and secured wherever it is most comfortable for the animal. When properly positioned, the collar gel pack should resemble the petals of a flower around the neck of the animal.

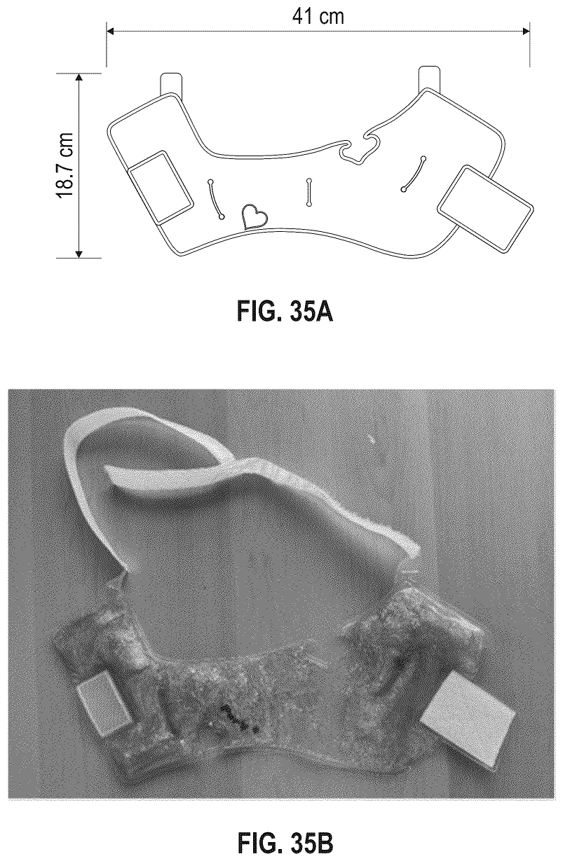

FIG. 35A is a schematic diagram of a muzzle gel pack for application to the snout (i.e., the nose, mouth, and whisker area) of a feline or canine animal and FIG. 35B is a photograph of the muzzle gel pack as manufactured according to the schematic diagram. The muzzle gel pack has application as a cold compress to ease pain following dental surgery, gum inflammation, mouth injuries, and tooth abscesses. The schematic diagram at FIG. 35A shows a 41 cm (16.2 in) length×18.7 cm (7.4 in) width×1 cm (0.4 in) height muzzle gel pack with two closures; however, it is to be understood that the muzzle gel pack may be manufactured in a variety of sizes to fit a variety of animals. The three voids shown in FIGS. 35A and 35B are integrated into the muzzle gel pack to allow the gel pack to be flexible to fit around the neck area of the animal. The muzzle gel pack is secured to the snout of the animal with two closures, a shorter closure that attaches at the underside of the jaw and a longer closure that attaches at the back of the neck (i.e., the dorsal neck area) of the animal.

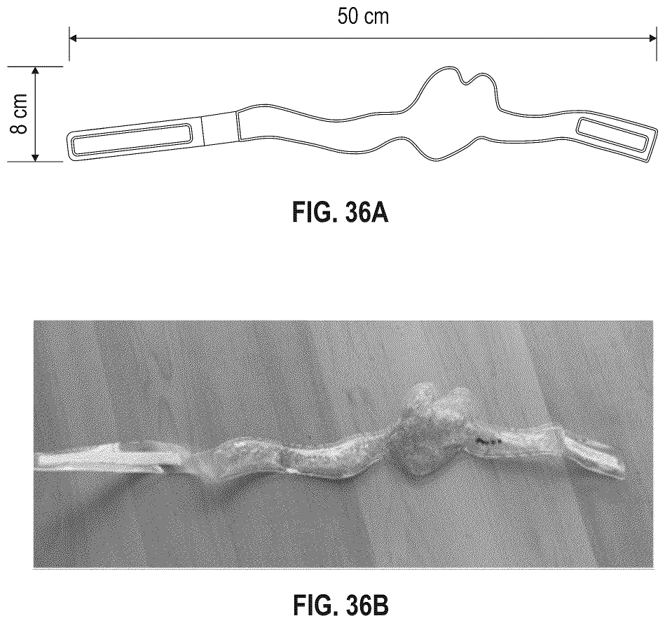

FIG. 36A is a schematic diagram of an eye gel pack for application to the eye area of a feline or canine animal and FIG. 36B is a photograph of the eye gel pack as manufactured according to the schematic diagram. The eye gel pack has application following eye surgery by reducing swelling, redness, inflammation, bruising, and pain around the suture site as well as providing a barrier to prevent the animal from scratching the surgical site. The schematic diagram at FIG. 36A shows a 50 cm (19.7 in) length×8 cm (3.2 in) width×1 cm (0.4 in) height eye gel pack; however, it is to be understood that the eye gel pack may be manufactured in a variety of sizes to fit a variety of animals. The eye gel pack will include one or two voids (not shown) to assist with the flexibility of gel pack. To apply the eye gel pack, the gel pack is wrapped around the animal's head and ears to cover the eyes and is secured at the back of the neck (i.e., the dorsal neck area) with a single long closure set.

In another embodiment, the gel pack for animals is a medical tape and/or a dressing pad gel pack wherein the medical tape and/or the dressing pad comprises oregano oil, almond oil, or a combination of oregano oil and almond oil. Oregano oil has been shown to have antibacterial, antiviral, antiparasitic, and antifungal properties; thus, the inclusion of oregano and/or almond oil to the medical tape and/or dressing pad gel packs may prevent the growth of harmful bacteria and/or microbes.

In another embodiment of the present invention, the therapeutic pack is an instant cold therapy pack. As is known in the art, an instant cold therapy pack is a disposable single-use cold compress that uses endothermic reaction to provide cold therapy without requiring prior refrigeration or freezing. A typical instant cold therapeutic pack is a pouch with two compartments, one compartment containing an endothermic salt and another compartment containing water, wherein the two compartments are separated by a thin breakable plastic barrier. As is known to those of skill in the art, an endothermic salt is a salt that absorbs heat from the environment when it dissolves in a solvent (typically water), causing the solution to become colder. Examples of endothermic salts include, without limitation, ammonium nitrate, urea, calcium ammonium nitrate, magnesium sulfate, and combinations thereof. When the instant cold gel pack is squeezed, the thin breakable plastic barrier between the two internal compartments ruptures causing the water and the endothermic salt to mix thus triggering an endothermic rection that lowers the temperature of the pack. Examples of plastic materials for the two compartments of the instant cold pack include, without limitation, polyethylene, polyethylene terephthalate, polypropylene, nylon, ethylene vinyl acetate, and combinations thereof. The thin breakable plastic barrier may be made of the same material as the pouch, but it will be significantly thinner such that it is able to rupture when the instant cold pack pouch is squeezed. Within the context of the present invention, the voids may be located in one or in both compartments in order to provide the instant cold therapy pack with the flexibility required to conform to an anatomical area or body part of a human or animal user. In order to ensure that the water and the endothermic salt thoroughly mix around the voids, the instant cold pouch may need to be gently shaken prior to use. It is to be understood that the instant cold therapy packs may include any of the shapes and void configurations described for the therapeutic gel packs.

In a further embodiment of the present invention, the therapeutic pack is an instant dry heat therapy pack. As is known in the art, an instant dry heat therapy pack is a disposable single-use heat compress that uses an exothermic reaction to provide immediate heat without the need for electricity, boiling, or microwaving. A typical instant dry heat therapy pack has a sealed outer packaging pouch that surrounds an air-permeable inner pouch that contains iron powder (reacts with oxygen to generate heat), salt (acts as a catalyst), activated charcoal (distributes heat), vermiculite or clay (provides heat retention), water (in very small amount to initiate the reaction). When the sealed outer packaging is opened, oxygen from the air enters into the air-permeable inner pouch and triggers an oxidation reaction with the iron powder, which releases heat-producing exothermic energy. The material of the outer sealed packaging for the instant dry heat therapy pack must be airtight and waterproof to prevent oxygen from entering the inner pouch prematurely. Examples of such airtight, waterproof materials include, without limitation, polyethylene, polyethylene terephthalate, aluminum foil, metallized film, and combinations thereof. Examples of materials for the air-permeable inner pouch include, without limitation, non-woven polypropylene (a breathable, durable, plastic-based fabric), spunbond fabric (a non-woven material made by bonding plastic fibers), microporous film, laminated paper, and combinations thereof. Within the context of the present invention, the inner pouch of the instant dry heat therapy packs will include the voids as described herein in order to provide the instant dry heat therapy pack with the flexibility required to conform an anatomical area or body part of a human or animal user. It is to be understood that the instant dry heat therapy packs may include any of the shapes and void configurations described for the therapeutic gel packs.

The descriptions of the various embodiments and/or aspects of the present disclosure have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the aspects and/or embodiments, the practical application or technical improvement over technologies found in the marketplace, or to enable others of ordinary skill in the art to understand the aspects and/or embodiments disclosed herein.

1. A gel pack comprising:
A gel material, and
a shell material enclosing the gel material,
wherein the gel pack is a cold or heat therapy pack that includes at least one void area free of the gel material, wherein the at least one void area provides the gel pack with flexibility that allows the gel pack to closely conform to an anatomical area or body part of a human or animal user.

2. The gel pack of claim 1, wherein the at least one void area comprises linear voids.

3. The gel pack of claim 1, wherein the at least one void area comprises non-linear voids.

4. The gel pack of claim 1, wherein the at least one void area comprises a combination of linear and non-linear voids.

5. The gel pack of claim 1, wherein the width of the at least one void area will range from 1-5 mm and the length of the at least one void area will range from 2-10 cm.

6. The gel pack of claim 1, wherein the shell material has a thickness of 1-2 mm without the gel material.

7. The gel pack of claim 1, wherein the thickness of the cold or heat therapy pack when filled with the gel material is 5-10 mm.

8. The gel pack of claim 1, wherein the gel material is a gel comprising a thickening agent, water, and an anti-freezing agent.

9. The gel pack of claim 8, wherein the thickening agent is selected from the group consisting of carboxymethyl cellulose, hydroxyethyl cellulose, sodium polyacrylate silica gel, and combinations thereof.

10. The gel pack of claim 8, wherein the anti-freezing agent is selected from the group consisting of tri-methylene glycol, diethylene glycol, ethylene glycol, propylene glycol, butanediol, glycerol, and combinations thereof.

11. The gel pack of claim 8, wherein the thickening agent is carboxymethyl cellulose and the anti-freezing agent is tri-methylene glycol.

12. The gel pack of claim 1, wherein the shell material is selected from the group consisting of polypropylene, polyvinyl chloride, polyethylene, polycarbonate, polytetrafluoroethylene, nylon, acrylonitrile butadiene styrene, and combinations thereof.

13. The gel pack of claim 1, wherein the shell material is polyvinyl chloride.

14. The gel pack of claim 1, wherein the gel pack is secured to the anatomical area or body part of the human or animal user with at least one set of closures.

15. The gel pack of claim 14, wherein the at least one set of closures are selected from the group consisting of hook and loop closures, buckle closures, strap closures, adhesive closures, and combinations thereof.

16. The gel pack of claim 1, wherein the shell material comprises a dressing pad on one side of the gel pack, wherein the side of the gel pack with the dressing pad is applied to a wound on the anatomical area or the body part of the human or animal user.

17. The gel pack of claim 16, wherein the gel pack is designed for application to an animal user, wherein the dressing pad is saturated with oregano oil and/or almond oil.

18. The gel pack of claim 1, wherein gel pack is designed for application to a human user, wherein the shell material comprises a medical grade silicone tape sheet on one side of the gel pack, wherein the side of the gel pack with the medical grade silicone tape sheet is applied to an area of a human user that requires scar maintenance.

19. The gel pack of claim 1, wherein the gel pack is designed to fit into a pocket of a compression garment, wherein the compression garment is designed to be worn around the anatomical area or the body part of the human or animal user.

20. The gel pack of claim 1, wherein the shell material comprises a pressure sensitive medical adhesive on one side of the gel pack, wherein the gel pack adheres to the anatomical area or the body part of the human or animal user via the medical adhesive.

21. The gel pack of claim 20, wherein the pressure sensitive medical adhesive is medical tape.

22. The gel pack of claim 20, wherein the pressure sensitive medical adhesive comprises a therapeutic agent selected from the group consisting of antibiotic agents, anti-inflammatory agents, pain relieving agents, anesthetics, and combinations thereof.

23. The gel pack of claim 22, wherein the therapeutic agent is the anti-inflammatory and pain-relieving agent known as Arnica montana.

24. The gel pack of claim 22, wherein the antibiotic agent is selected from the group consisting of neomycin, bacitracin, polymyxin, and combinations thereof.

25. The gel pack of claim 21, wherein the gel pack is designed for application to an animal user, wherein the medical tape comprises oregano oil and/or almond oil.

26. An instant cold therapy pack comprising a plastic pouch with a first compartment containing an endothermic salt and a second compartment containing water, wherein the first and second compartments are separated by a thin plastic barrier, wherein squeezing of the pouch ruptures the thin plastic barrier causing the water and endothermic salt to mix and form an endothermic reaction that lowers the temperature of the therapeutic pack, wherein the first and/or second compartments of the pouch comprise at least one void area free of the water and/or the endothermic salt, wherein the at least one void area provides the instant cold therapy pack with flexibility that allows the instant cold therapy pack to closely conform to an anatomical area or body part of a human or animal user.

27. An instant dry heat therapy pack comprising an airtight and waterproof sealed outer pouch and an air-permeable inner pouch, wherein the air-permeable inner pouch comprises a filling that includes iron powder, wherein unsealing of the outer pouch exposes the iron powder to air, wherein reaction of the iron powder and oxygen from the air forms an oxidation reaction that raises the temperature of the therapeutic pack, wherein the air-permeable inner pouch comprises at least one void area free of the filling, wherein the at least one void area provides the instant dry heat therapy pack with flexibility that allows the instant dry heat therapy pack to closely conform to an anatomical area or body part of a human or animal user.
